(19) United States
(12) Patent Application Publication
(10) Pub. No.: US 2005/0049505 A1
(43) Pub. Date: Mar. 3, 2005

(54) INTRAVENOUS INJECTION DEVICE

(76) Inventors: Chung-Yuo Wu, Taipei Hsien (TW); Meng-Tsung Lo, Taipei Hsien (TW); Yi-Chung Chang, Taipei Hsien (TW)

Correspondence Address: (NAIPC) NORTH AMERICA INTERNATIONAL PATENT OFFICE, P.O. BOX 506, MERRIFIELD, VA, 22116, US

(21) Appl. No.: 10/708,397

(22) Filed: Feb. 29, 2004

(30) Foreign Application Priority Data
Aug. 27, 2003 (TW) 092123594

(51) Int. Cl. A61B 8/02

(52) U.S. Cl. 600/455

(57) ABSTRACT

An intravenous injection device for detecting the position of a vein of an examinee includes a pedestal comprising a housing, a pulse ultrasound probe installed in front of the housing, and a microprocessor installed in the housing wherein the pulse ultrasound probe includes an oscillator for emitting a pulse ultrasonic signal toward the examinee along the direction of the housing and a sensor for receiving ultrasonic signals reflected by the examinee and converting the reflected signals into electric signals to output to the microprocessor, a propeller for moving the pedestal along the direction of the pulse ultrasonic signals, and a syringe connected to and conveyed by the propeller to move along the direction of the pulse ultrasonic signals.

### BACKGROUND OF INVENTION

1. Field of the Invention

The invention relates to an intravenous injection device, and more particularly to an intravenous injection device utilizing an ultrasonic vein detector.

2. Description of the Prior Art

Generally performed medical services such as endoscope operations or radiation tumor treatments are accompanied with objective data-gathering means such as X-ray scan or magnetic resonance imaging (MRI) in order to provide objective images to the operator in order to aid in the operation.

On the other hand, blood tests and injections depend only on the experience of operators in selecting a proper position to insert the needle to extract a blood sample or inject medicine; no objective references are used. In the case of the examinee being the fat or the infant, the task is made more difficult because their vein is not always easily found, meaning that the operators may have to repeatedly insert the needle until the proper position for injection is found. That not only causes pain to the examinee but also raises the risk. Therefore, an objective reference of the position of veins is necessary to increase the quality of medical cares.

However, the reflection rates of blood and soft tissues do not differ so much from each other. Therefore, it is difficult to distinguish the blood and soft tissues by using the reflective signals. FIG. 1 shows a conventional ultrasound probe detecting blood current. The conventional ultrasound probe uses the method of the Doppler effect. When a blood cell 80 moves toward an ultrasonic emitter 91 as in FIG. 2, the frequency of the reflected signal received by a sensor 92 will become a little higher than the frequency of the signal sent by the emitter 91 and when the blood cell 80 moves away from the ultrasonic emitter 91 as in FIG. 3, the sensor 92 will sense a signal with lower frequency. Such phenomenons are also known as Doppler effect. Therefore, FIG. 4 shows the electrocardiogram (upper) and a Doppler shift blood current diagram (lower) measured by the prior art. The horizontal axis means time axis. It is obvious that they are related.

However, it is required to select a vein with low current speed for blood test or injection, meaning that the Doppler shift effect is generally unobvious; added, it is difficult to distinguish the blood from neighboring soft tissues. Furthermore, continuous ultrasonic detecting can only tell the operators whether there is a moving object but cannot provide any axial analysis, i.e. the depth of the moving object remains unknown. Therefore, it is still an object to improve the conventional technology for a better medial care.

FIG. 5 shows a device disclosed by US Published Applications No. 20020133079 that utilizes a probe 72 to detect the position of a blood vessel, and a horizontal guiding holder 74 to hold a syringe 73, so that the operator can insert the syringe 73 into a proper position according to the data provided by the device.

However, the angle between the holder 73 of the syringe 74 and the direction of the ultrasonic signal generated by the ultrasound probe 72 is large. It means that if the syringe 73 is inserted at the incorrect angle, the size of the error becomes larger according to the depth of insertion, and cannot be corrected unless the syringe 73 is reinserted. Moreover, the syringe 73 is positioned so far from the probe 72 that it is not convenient for operators to hold them with two hands.

Therefore, a low-cost high-reliable device providing data on depth according to the reflective ultrasonic signals in order to find a proper position of injection and especially capable of being held with one hand is necessary.

### SUMMARY OF INVENTION

It is therefore a primary objective of the claimed invention to provide an ultrasonic intravenous injection device capable of injecting medicine correctly. The device according to the present invention is low-cost, automatic, and capable of being operated with one hand.

Briefly, an intravenous injection device for detecting the position of a vein of an examinee includes a pedestal including a housing, a pulse ultrasound probe installed in front of the housing, and a microprocessor installed in the housing wherein the pulse ultrasound probe includes an oscillator for emitting a pulse ultrasonic signal toward the examinee along the direction of the housing and a sensor for receiving ultrasonic signals reflected by the examinee and converting the reflected signals into electric signals to output to the microprocessor, a propeller for moving the pedestal along the direction of the pulse ultrasonic signals, and a syringe connected to and conveyed by the propeller to move along the direction of the pulse ultrasonic signals.

The present invention utilizes the pulse ultrasonic oscillator to measure the reflective signals to accumulate effective reflection according to the Doppler's effect in order to obtain the data on the depth of a vein and then uses the propeller to insert the syringe at a proper depth along the signal direction so that the precision and automation are both improved.

These and other objectives of the present invention will no doubt become obvious to those of ordinary skill in the art after reading the following detailed description of the preferred embodiment that is illustrated in the various figures and drawings.

### BRIEF DESCRIPTION OF DRAWINGS

FIG. 1 illustrates a conventional ultrasound probe detecting blood current.

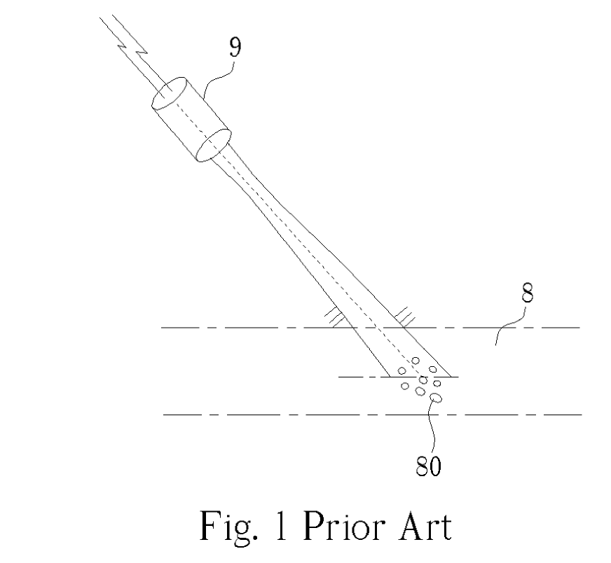

FIG. 2 illustrates an ascending shift of the reflective ultrasonic signals according to the Doppler effect when the object moves toward the ultrasound probe.

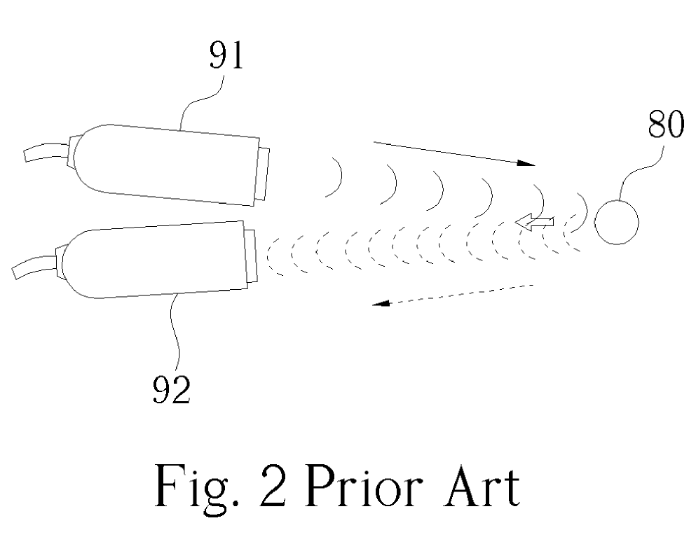

FIG. 3 illustrates descending shift of the reflective ultrasonic signals according to the Doppler effect when the object moves away from the ultrasound probe.

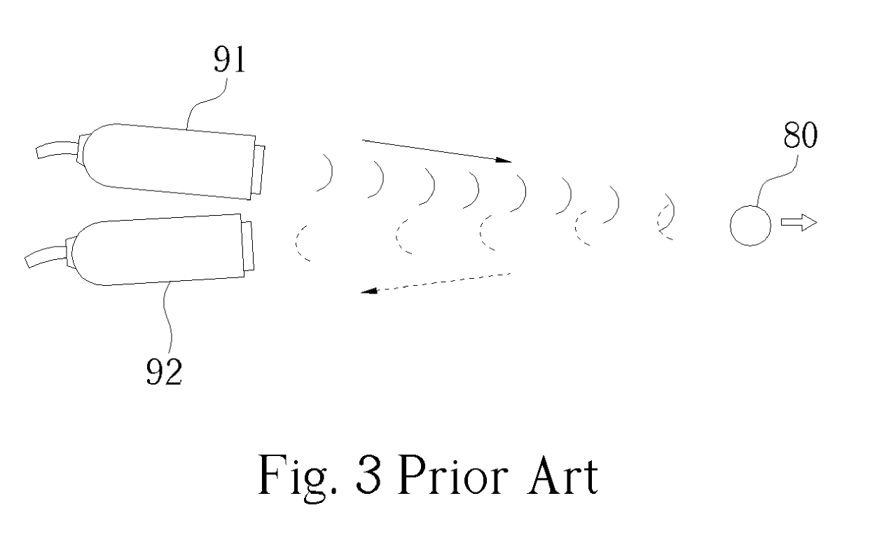

FIG. 4 is a timing diagram comparing the Doppler shift of the blood current measured by the ultrasonic signals with an electrocardiogram.

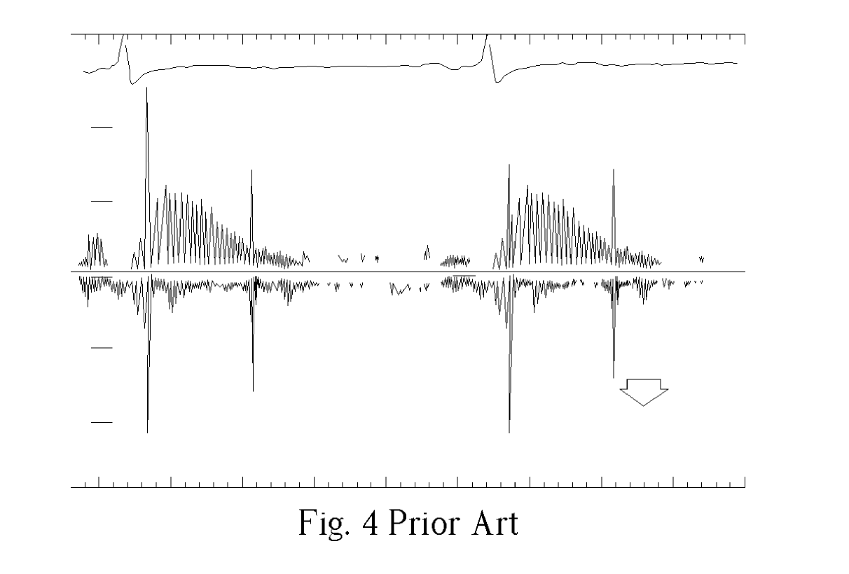

FIG. 5 illustrates the device according to US Published Applications No. 20020133079 filed in 2002.

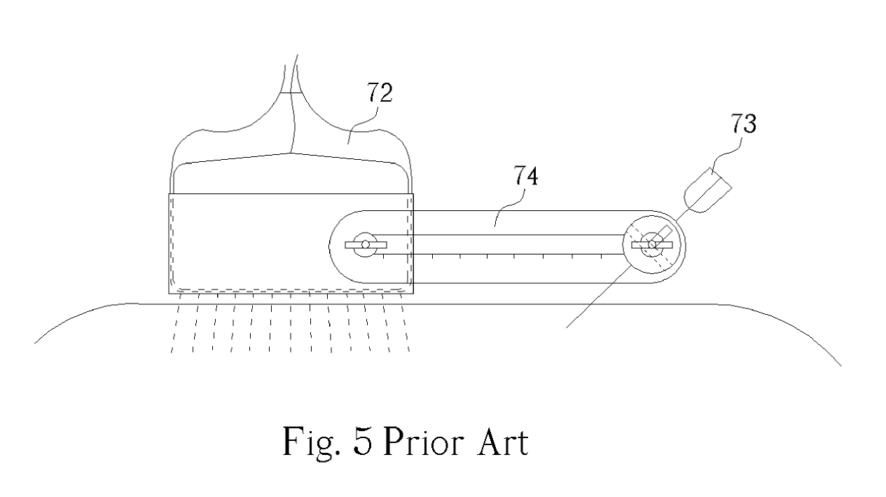

FIG. 6 illustrates the first embodiment of the present invention.

FIG. 7 is a block diagram of the first embodiment.

FIG. 8 illustrates the second embodiment of the present invention.

### DETAILED DESCRIPTION

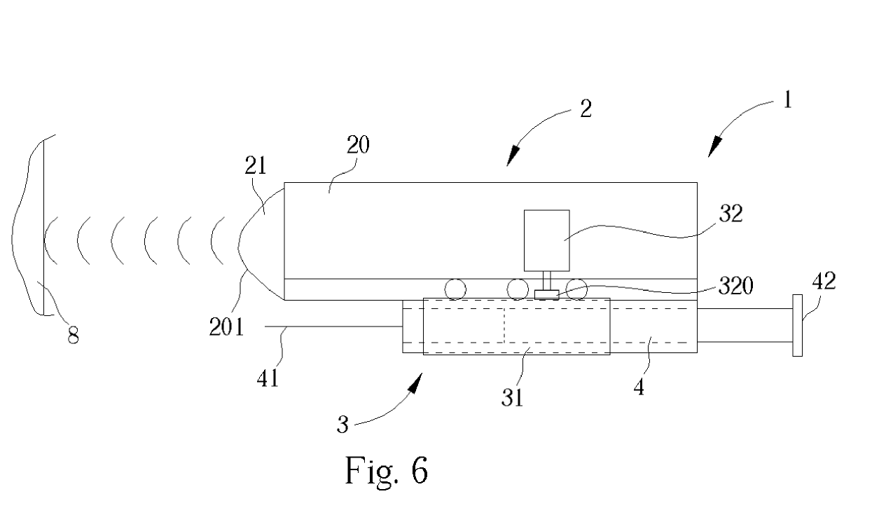

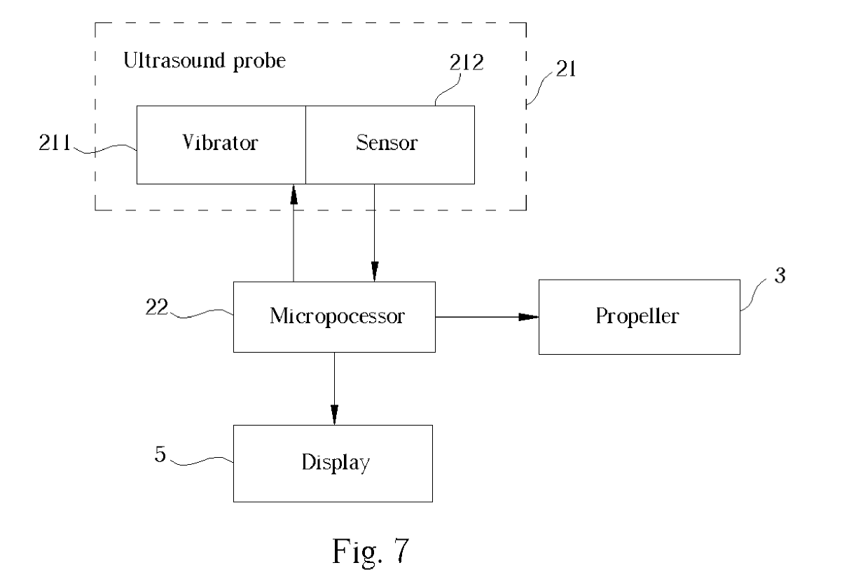

Please refer to FIG. 6 and FIG. 7; an intravenous injection device 1 according to the present invention includes a pedestal 2, a propeller 3 and a syringe 4.

The pedestal 2 includes a housing 20, a pulse ultrasound probe 21 contained in the housing 20, and a microprocessor 22. The end of the housing 20 toward the examinee is hereby defined as a front end 201. In order not to be interfered with, the probe 21 is installed at the front end 201. The probe 21 includes an oscillator 211 and a sensor 212. In the present embodiment, when the microprocessor 22 generates a command to the oscillator 211, the oscillator 211 oscillates, thereby generating pulse ultrasonic signals that it forwards. The signals are reflected by interfaces between different tissues and organs and then received by the sensor 212 to be converted into electrical signals to be output to the microprocessor 22.

Since the ultrasonic signals are pulse signals, after a period of time, the reflective volume can be calculated according to the Doppler effect in order to analyze a time interval of the signal reflection and the signal emission, and the resulting time interval is multiplied by the transmission speed of the ultrasonic signal to calculate the correct data on the depth of the vein. The data is then output to a display 5 for the examiner.

The propeller 3 in this embodiment includes a clipper 31 and a motor 32 fixed to the housing 20. The motor 32 has a power output 320 in contact with the clipper 31. Driven by a driving signal of the microprocessor, the motor can move the clipper 31 back and forth along the direction of the parallel pulse ultrasonic signals.

The syringe 4 is clipped by the clipper 31, and the needle 41 of the syringe 4 points ahead. When the sensor 212 receives the reflected signals from the blood in the vein and the microprocessor 22 analyzes the data on the depth of the vein, the examiner only needs to push a button (not shown). Then the microprocessor 22 generates the driving signal to activate the motor 32 in order to move the clipper 31 forward so that the syringe 4 clipped by the clipper 31 is then inserted into the examinee with the depth of insertion being controlled by the microprocessor 22. In this embodiment, the depth limit is 2 cm, and the needle 41 can be stopped when it approaches the limit. In such a manner, the needle 41 is precisely inserted into the vein, and the examiner only needs to push (or pull) a plug 42 of the syringe 4 to inject medicine into (or extract blood from) the vein. Of course, as known by the person skilled in the art, the motor can also be installed on the clipper 31 with its power output 320 in contact with the housing 20 in order to move the pedestal 2 back and forth. Such kind of a design also belongs to the present invention.

Since the direction of the syringe 4 is mostly parallel to the housing 20 and the signal direction, the data on depth obtained by sensing reflected ultrasonic signals is less distortion, and the precision and automation are accordingly improved.

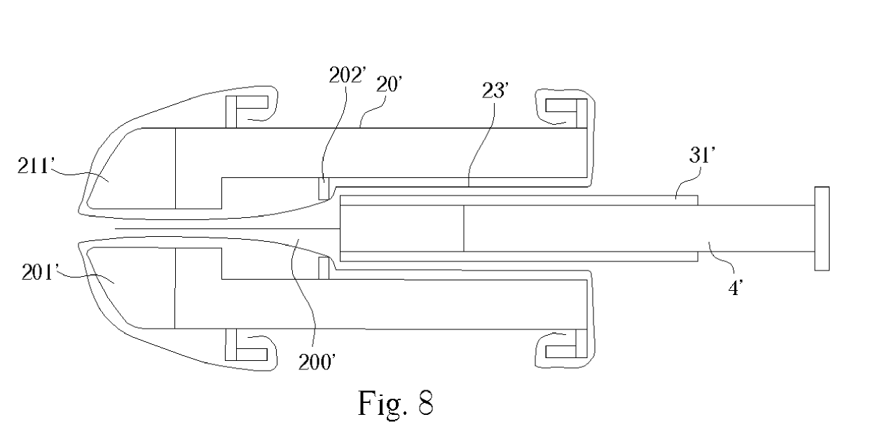

Of course, as known by the person skilled in the art, in order to reduce the distance between the signal direction and the moving direction of the syringe thereby and the error on the deviated angle, a second embodiment of the present invention is disclosed in FIG. 8. An aperture 200 is formed on a housing 20 of a pedestal 2, and a clipper 31 is contained in the aperture 200. If the aperture 200 is on the center of the housing 20 and the ultrasonic oscillator 211 is installed in a circle at a front end 201 of the housing 20, the data on depth will overlap the moving axis of the clipper 31 without any deviation.

In this embodiment, the pedestal 2 further includes a disposable cover 23 covering the front end 201 and the inner wall of the housing 20 to be disposed every time after injection or extraction in order to prevent blood contamination. A stopper 202 is formed on the inner wall of the housing 20 to stop the syringe 4 at a predetermined depth limit such as 2 cm.

In contrast to the prior art, the intravenous injection device according to the present invention utilizes a pulse ultrasonic oscillator in detection along a specific direction to obtain the data of the depth of the vein by accumulating effective reflection via the Doppler effect. Accompanied by the propeller conveying the syringe to be inserted into the vein of the examinee, the present invention can be applied even if the examinee is the fat or the infant. Moreover, by connecting the ultrasonic oscillator with the injecting device, the examiner can operate it with only one hand.

Those skilled in the art will readily observe that numerous modifications and alterations of the device may be made while retaining the teachings of the invention. Accordingly, the above disclosure should be construed as limited only by the metes and bounds of the appended claims.

1. An intravenous injection device for detecting the position of a vein of an examinee and injecting comprising:
a pedestal comprising a housing, a pulse ultrasound probe installed in front of the housing, and a microprocessor installed in the housing wherein the pulse ultrasound probe comprises an oscillator for emitting a pulse ultrasonic signal toward the examinee along the direction of the housing and a sensor for receiving the ultrasonic signals reflected by the examinee and converting the reflected signals into electric signals to output to the microprocessor;
a propeller for moving the pedestal along the direction of the pulse ultrasonic signals; and
a syringe coupled to the propeller, being moved along the direction of the pulse ultrasonic signals by the propeller.

2. The intravenous injection device of claim 1 wherein the propeller is driven by a driving signal from the microprocessor.

3. The intravenous injection device of claim 1 wherein the propeller comprises a clipper for clipping the syringe, and a motor fixed on the housing or the clipper and its power output coupled to the clipper or the housing in order to convey the clipper along the direction of the pulse ultrasonic signals.

4. The intravenous injection device of claim 3 wherein an aperture is formed on the housing to contain the clipper.

5. The intravenous injection device of claim 4 further comprising a cover covering the front end of the housing and the inner wall of the housing to prevent the intravenous injection device from contaminating.

6. The intravenous injection device of claim 4 wherein a stopper is formed at a predetermined distance from the front end on the inner wall of the housing in order to stop the clipper at a predetermined depth.
